(19) United States
(12) Patent Application Publication
(10) Pub. No.: US 2022/0128573 A1
(43) Pub. Date: Apr. 28, 2022

(54) BIOMARKERS FOR DIAGNOSIS OF OCULAR DISEASES AND THE METHOD THEREOF

(71) Applicant: Narayana Nethralaya Foundation, Bangalore (IN)

(72) Inventors: Arkasubhra GHOSH, Bangalore (IN); Rohit SHETTY, Bangalore (IN); Narendra P, Bangalore (IN); Swaminathan SETHU, Bangalore (IN)

(73) Assignee: Narayana Nethralaya Foundation, Bangalore (IN)

(21) Appl. No.: 17/430,490

(22) PCT Filed: May 30, 2019
(86) PCT No.: PCT/IB2019/054472

§ 371 Date: Aug. 12, 2021

(30) Foreign Application Priority Data
Feb. 13, 2019 (IN) 201941005765

(51) Int. Cl. G01N 33/68

(52) U.S. Cl. CPC G01N 33/6893; G01N 2333/475; G01N 2333/525; G01N 2800/164; G01N 2333/705; G01N 2333/90633; G01N 2333/95; G01N 2333/5421

(57) ABSTRACT

The present invention relates to the quantification of various biomarkers, including MMP9 (Inflammatory marker), LOX (Lysyl oxidase), IL6 (Interleukin-6), TNFα (tumor necrosis factor alpha), VEGF (Vascular Endothelial Growth Factor) ICAM-1 (Intercellular Adhesion Molecule-1) in aqueous humor, vitreous humor, tear and serum of patients with ocular diseases. The invention further describes the role of biomarkers in the pathogenesis, progression of ocular diseases. Hence, the level of biomarkers serves as a diagnostic and/or prognostic marker in ocular diseases. The invention further relates to the use of multiple biomarkers that can be simultaneously used for testing various corneal and retinal diseases.

### DESCRIPTION OF THE INVENTION

### Technical Field of the Invention

The present invention relates to the association observed between biomarkers and ocular diseases. Particularly, the invention relates to the association between biomarkers such as Matrix metallopeptidase 9 (MMP-9), lysyl oxidase (LOX), TL6, Tumor Necrosis Factor-α (TNFα), Vascular Endothelial Growth Factor (VEGF), Intercellular Adhesion Molecule-1 (ICAM1) and Interleukin-6 (IL6) and ocular diseases such as Keratoconus (KC), Dry eye disease (DED), Corneal inflammation, Corneal ectasia patient stratification and

Anti VEGF therapy monitoring for retinal vascular diseases such as Diabetic Retinopathy (DR), Age-related macular degeneration (AMD), Vein occlusion and retinal inflammation. The invention further relates to the use of multiple biomarkers that can be simultaneously used for testing various corneal and retinal diseases.

### BACKGROUND OF THE INVENTION

Ocular inflammatory symptoms are associated with a wide variety of disorders including Dry Eye Disease (DED), Keratoconus (KC), Corneal Ectasia and Ocular surface infections. The overt symptoms of inflammation observed by direct examination of the ocular surface are often similar across the diseases and do overlap across the conditions sometimes resulting in misdiagnosis, negligence or empirical treatments. Thus, it is often challenging for primary care physicians to arrive at an accurate diagnosis and pin point the underlying primary cause of the ocular disease in the absence of specialized diagnostic devices which are available only at large tertiary care ophthalmic institutions. In addition, post-refractive surgery corneal ectasia in healthy eyes is a major complication which is difficult to predict prior to surgery.

Keratoconus, a degenerative condition of the front of the eye is particularly difficult to diagnose even for practicing specialist clinicians, since the inflammatory symptoms are not always severe. Furthermore, allergies and systemic inflammatory conditions can also confound diagnosis. In addition, thinning or ectasia of the cornea may happen after refractive surgery, which is performed in thousands of eyes every year. Molecular factors in the cornea often predispose subjects to developing this major complication after refractive surgery which currently do not have a simple way of being tested for surgeons to select patients stringently.

It is estimated that 1 to 430/2000 general population are affected by KC and similarly, a high number with a wide range from 7.8% and sometimes >30% of population are affected by DED Inflammatory conditions of the eye such as KC, DED can be more effectively managed with excellent prognosis if identified accurately at an early stage. Hence, diagnostic methods that can help in early diagnosis, patient stratification, patient selection and monitoring therapy responses will be of immense use.

Ocular surface is prone for both viral and bacterial infections and it is necessary to have accurate and early diagnosis because misdiagnosis results in the spread of disease and unnecessary use of antibiotics. Viral infections of the ocular surface such as adenovirus, influenza and bacterial infections such as Chlamydia trachomatis require easy, point-of-care and accurate detections systems. Adenoviral keratoconjunctivitis affect between 15% and 20% of the population 3 and ocular C. trachomatis infection is known to be one of the leading infectious causes of blindness.

Diabetic retinopathy is leading cause of blindness in adults and it is estimated that there are greater than 90 million people suffering from DR. Vascular endothelial growth factor (VEGF) is a major factor that contributes to neovascularization resulting in DR and hence serves as both target and a biomarker for DR. Diabetic retinopathy is the most common ocular complication of diabetes occurring in 15% of individuals suffering from diabetes, and often can result in blindness. Determining if a patient is appropriate for multiple anti-VEGF therapy injections or for intravitreal steroids is an urgent need for retina physicians.

The US Patent Application US20170131291A1 titled “Methods and devices for diagnosing ocular surface inflammation and dry eye disease” discloses a method for diagnosis and prognosis of Dry Eye Disease using biomarkers. It also discloses a kit which can be used for rapid diagnosis and monitoring of subject for Dry eye disease and monitoring the therapy by using a panel of biomarkers. However, the invention does not mention the use of a single method for diagnosing more than one corneal or retinal disease. There is no reference to MMP9, LOX, IL6 and TNF alpha as biomarker for corneal disease, or ICAM1, VEGF, IL6 and TNF alpha as biomarker for retinal diseases. Further the sample type is restricted to tear hence other sample types like aqueous humor, vitreous humor or serum are not included.

The Patent Application US20170248573A1 titled “Systems and methods for integration of microfluidic tear collection and lateral flow analysis of analytes of interest” discloses devices and methods for analyzing small volumes of biological samples. The method described in the invention pertains to use of less than twenty microliter of sample and detecting and measuring the concentration of one or more analytes in the fluid sample. However, there is no reference to a biomarker for corneal disease or biomarker for retinal diseases. Further the sample type is restricted to tear hence other sample types like aqueous humor, vitreous humor or serum are not included.

The Patent Application US20150005186A1 titled “Methods and devices for classifying and managing autoimmune and inflammatory conditions” discloses a diagnostic method for differentiating the immunopathological mechanism in patients having an autoimmune or inflammatory condition using suitable biomarkers. The biomarker species include IL-1beta, IL-1alpha, IL-1Ra, IL-15, IL-7, IL-2, IL-3, IL-4, IL-5, IL-6, GM-CSF, IL-18, IL-8, IL-12p70, IL-12p40, IL-17, IL-23, CXCL-10, MMP-9, ICAM-1, MIP-1alpha, MIP-1 beta, Complement 3, alpha1-antitrypsin, apolipoprotein A1, apolipoprotein CIII, and IgM. The invention further mentions the use of antigen-antibodies and performing a multiplex immunoassay. However, the invention does not mention the use of a single kit for diagnosing more than one corneal or retinal disease. There is no reference to LOX, VEGF and TNF alpha as biomarker for eye diseases. Further the invention mentions tear as preferable sample type and hence other sample types like aqueous humor, vitreous humor or serum is not considered.

There are different methods available for the quantification of biomarkers. The quantification of biomarkers is crucial in understanding the relationship between the biomolecules in the biological fluids and disease pathogenesis. Hence, quantification of biomarkers determines the relationship between the levels of biomarkers and stage/progression of disease. It can also serve as diagnostic and prognostic markers for various conditions including ocular diseases.

### SUMMARY OF THE INVENTION

The present invention relates to quantification of various biomarkers such as MMP9 (Inflammatory marker), LOX, IL6, TNFα, VEGF (Vascular Endothelial Growth Factor) ICAM-1 (Intercellular Adhesion Molecule-1) in tear, aqueous humor and vitreous humor which helps in diagnosis and in determining the prognosis of ocular diseases such as dry eye disease (DED), retinal angiogenic diseases Diabetic Retinopathy (DR), Age-related macular degenetation (AMD), retinal vein occlusive diseases (CRVO, BRVO, CRAO and BRAO), Dysfunctional tear syndromes (DTS), predisposition to Corneal Ectasia and Keratoconus (KC).

The invention utilizes a method of quantitative immunodetection of multiple biomarkers for ocular diseases. A panel of antibody-based biomarkers such as LOX, MMP9, IL6 and TNFα for testing corneal disease and ICAM1, VEGF, IL6 and TNFα are used for testing retinal disease. The detection methods utilizes multiplex platforms wherein the multiple analytes are measured simultaneously in the sample. In addition, the analyte measurement method provides direct quantification or semi-quantitative tiled readouts to classify the subject and aid in clinical decisions.

The comparative levels of LOX, MMP9, IL6, TNFα, ICAM1, VEGF, IL6 and TNFα are quantified by the invention method. The sample is withdrawn from the patients target area such as tear, aqueous humor, vitreous humor and serum. The results indicate that the levels of MMP-9, IL-6, ICAM1, MCP-1, IL-17F and VEGF increases in patients with KC, DED and DR disease and there is a loss of LOX in case of KC.

### BRIEF DESCRIPTION OF THE DRAWINGS

The foregoing and other features of embodiments will become more apparent from the following detailed description of embodiments when read in conjunction with the accompanying drawings.

FIG. 1 illustrates the levels of MMP-9, IL6 and TNFα in the tears of KC patients with different grades of KC.

FIG. 2 illustrates the levels of IL-6 in the tears of KC patients with different grades of KC.

FIG. 3 illustrates the levels of TNFα in the tears of KC patients with different grades of KC.

FIG. 4 illustrates the reduced levels of LOX activity in the tears of KC patients.

FIG. 5 illustrates the increased levels of indicated interleukins, cytokines, chemokines and other soluble factors in the tears of DED patients.

FIG. 6 illustrates the VEGF in the aqueous humor of diabetic patients without vision problems and diabetic retinopathy patients.

### DETAILED DESCRIPTION OF THE INVENTION

In order to make the matter of the invention clear and concise, the following definitions are provided for specific terms used in the following description.

The term “Diagnostic Biomarker” refers to a chemical/biological/physical entity which by means of specific reactions assists in qualitative and quantitative analysis of the disease.

The term “Prognostic Biomarker” refers to a chemical/biological/physical entity that provides information about the status of a specific disease in either untreated or treated individuals with the aim of determining the course of the condition or therapeutic response thereof.

The present invention relates to quantification of various biomarkers such as of LOX, MMP9, IL6, TNFα, ICAM1, VEGF, IL6 and TNFα in aqueous humor, vitreous humor, tear and plasma, which helps in diagnosis and also in determining the prognosis of various ocular diseases.

The invention relates to identification of biomarkers as diagnostic or prognostic markers in analysis of progression of the ocular diseases. The biomarkers are analyzed in fluids such as aqueous humor, vitreous humor, tear and serum of patients with ocular diseases.

The invention utilizes any existing method for quantitative immunodetection of multiple biomarkers for corneal and retinal diseases. The sample extracted from the fluids obtained from patients is added to the sample input area. The biomarkers in the sample complex are measured with the detection probe and associated hardware. The estimated levels of the biomarkers are compared to established standards and a quantitative or semi-quantitative readout as provided by the measurement device.

FIG. 1 illustrates the levels of MMP-9 IL6 and TNFα in the tears of KC patients with different grades of KC. Tear protein levels of MMP9, IL6 and TNFα correlate with KC and progression of the disease.

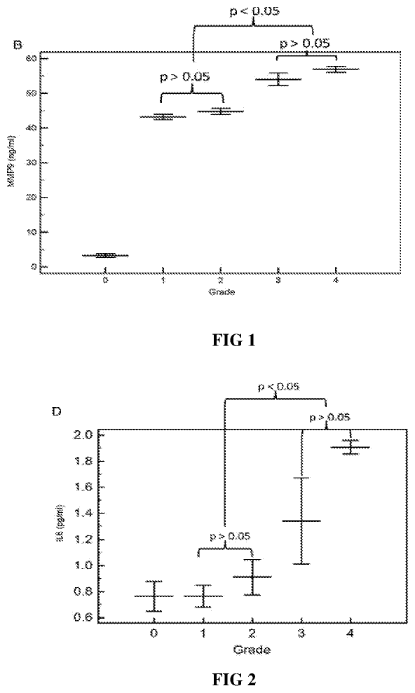

FIG. 2 illustrates the levels of IL-6 in the tears of KC patients with different grades of KC. The levels of IL-6 are increased in patients and correlated with the progression of the disease.

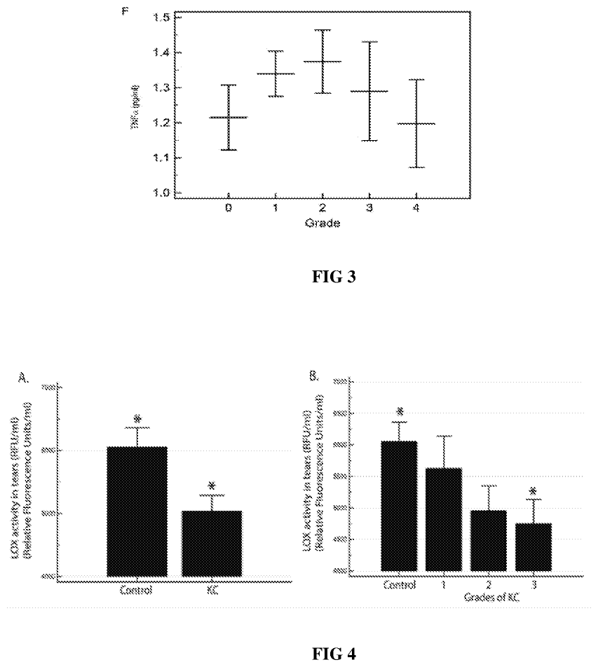

FIG. 3 illustrates the levels of TNFα in the tears of KC patients with different grades of KC. The levels of TNFα in tears are increased in patients in KC and correlated with the progression of the disease.

FIG. 4 illustrates the reduced levels of LOX activity in the tears of KC patients. The LOX activity in tears is reduced in patients in KC.

FIG. 5 illustrates the increased levels of indicated interleukins, cytokines, chemokines and other soluble factors in the tears of DED patients.

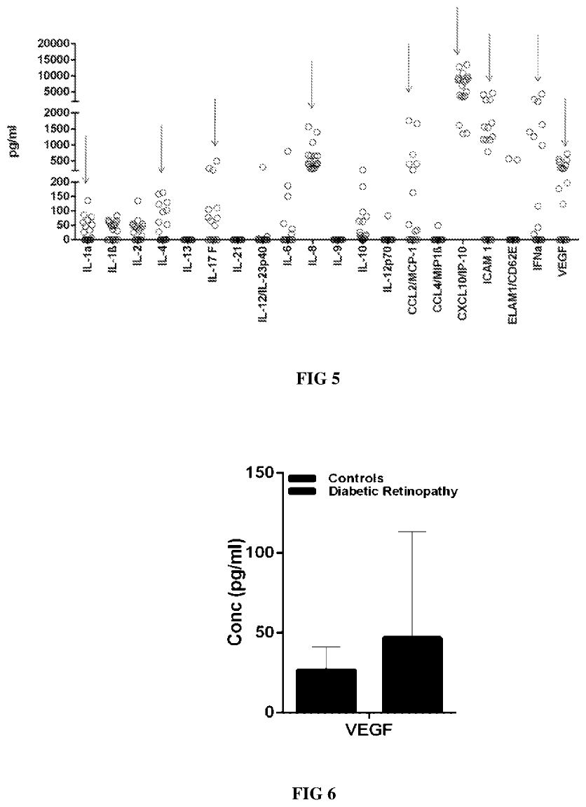

FIG. 6 illustrates the VEGF in the aqueous humor of diabetic patients without vision problems and diabetic retinopathy patients. However, the elevated VEGF levels are treated using anti-VEGF therapy.

The invention thus describes that higher levels of biomarkers are found in aqueous humor, vitreous humor, tear and plasma of patients with ocular diseases. Biomarkers levels are altered (higher or lower) in the fluid samples of patients with corneal and retinal diseases. Hence, indicating a vital role of biomarkers in the pathogenesis and progression of ocular diseases.

We claim:

1. Biomarkers to identify the risk of ocular diseases, wherein:
a. the biomarkers are quantified to be used to diagnose and prognosticate ocular diseases; and
b. the biomarker is quantified in a fluid of a human.

2. The biomarker as claimed in claim 1, wherein the biomarker is selected from a group comprising MMP9 (Inflammatory marker), LOX (Lysyl oxidase), IL6 (Interleukin-6), TNFα (tumor necrosis factor alpha), VEGF (Vascular Endothelial Growth Factor) ICAM-1 (Intercellular Adhesion Molecule-1).

3. The biomarker as claimed in claim 1, wherein the fluid is selected from a group comprising tear, aqueous humor, vitreous humor and serum.

4. The biomarker as claimed in claim 1, wherein the ocular conditions are selected from a group comprising of dry eye disease (DED), retinal angiogenic diseases Diabetic Retinopathy (DR), Age-related macular degenetation (AMD), retinal vein occlusive diseases (CRVO, BRVO, CRAO and BRAO), Dysfunctional tear syndromes (DTS), predisposition to Corneal Ectasia Dysfunctional tear syndromes (DTS), and Keratoconus (KC).

5. The biomarker as claimed in claim 1, wherein the levels of biomarkers are high in patient with ocular diseases.
